(19) United States
(12) Patent Application Publication
(10) Pub. No.: US 2025/0093637 A1
(43) Pub. Date: Mar. 20, 2025

(54) PROXIMITY SENSOR FOR DIGITAL OCULAR SYSTEM

(71) Applicant: Alcon Inc., Fribourg (CH)

(72) Inventor: Mauricio Jochinsen, Fountain Valley, CA (US)

(73) Assignee: Alcon Inc., Fribourg (CH)

(21) Appl. No.: 18/761,871

(22) Filed: Jul. 2, 2024

Related U.S. Application Data
(60) Provisional application No. 63/582,614, filed on Sep. 14, 2023.

(51) Int. Cl. G02B 21/36; G02B 21/00; G02B 21/02; G02B 25/00

(52) U.S. Cl. CPC G02B 21/368; G02B 21/0012; G02B 21/02; G02B 25/001

(57) ABSTRACT

A digital ocular system includes a housing, an eye piece having a lens assembly. A display screen, a proximity sensor, and a processor. The lens assembly includes a front lens through which a user of the digital ocular system views a target object. The display screen is positioned within the housing cavity. The proximity sensor, which is connected to the housing, detects when user is within a predetermined standoff distance of the front lens, and outputs an electronic sensor signal when the user is outside of the predetermined standoff distance, i.e., not detected. The processor is configured to adjust an output state of the display screen via a display control signal in response to the electronic sensor signal.

### CROSS-REFERENCE TO RELATED APPLICATIONS

The present application claims the benefit of priority to U.S. Provisional Application No. 63/582,614 filed on Sep. 14, 2023, which is hereby incorporated by reference in its entirety for all purposes.

### INTRODUCTION

The present disclosure relates to automated systems and methods for preventing image “burn-in” of a display screen of an ophthalmic visualization system equipped with digital oculars.

Digital and hybrid camera-equipped microscopes are able to acquire digital pixel images of a magnified target object. The acquired images are often displayed via a monitor or display screen. In a three-dimensional (3D) ophthalmic “heads-up” visualization system, for instance, one or more such display screens are situated within a surgical suite within ready view of the clinician and attending medical staff. In this exemplary use scenario, the display screens present a magnified view of a patient's ocular anatomy in real-time. The displayed images are then viewed through special polarized glasses worn by the clinician and staff to allow the viewers to enjoy a 3D viewing effect.

Medical display screens of the type used in the above-noted surgical suite are often configured as organic light-emitting diode (OLED) screens. As appreciated in the art, each constituent pixel of an OLED screen is individually illuminated rather than relying on a common backlight source. As a result of this and other technical features of OLED display screens, medical display screens tend to be far brighter and produce clearer images relative to other LED-driven display screens. However, OLED screens and other LED-based medical display screens remain prone to the above-noted burn-in phenomenon, during which permanent ghost-like images result from prolonged static image display and lighting source use/aging.

### SUMMARY

Systems and methods are disclosed herein for preventing “burn-in” of a medical display screen that is contained within a housing of a digital ocular device. Digital oculars include one or more eye pieces. Each eye piece has a front lens through which a clinician or other user of a microscope views a target object, e.g., a patient's eye in a representative ophthalmic surgical context. The digital oculars could be optionally configured as digital binoculars having two such eye pieces. Each respective front lens of the digital oculars is positioned relative to a medical display screen, e.g., an organic light-emitting diode (OLED) screen, with each display screen wholly contained within a housing of the aforementioned digital oculars.

As part of the solutions to burn-in disclosed herein, a proximity sensor is connected to the housing of the digital oculars. The proximity sensor could be connected to an outer surface of the housing in one or more embodiments, or within a cavity defined by the housing. The latter configuration would provide added protection from interference with objects in the surgical suite such as surgical drapes and other external obstructions. The proximity sensor for its part is configured to detect when the user is within a predetermined eye relief distance (“standoff distance”) from the front lens(es) of the eye piece, e.g., within about 25-30 millimeters (mm) in a possible implementation. When the user is not within the standoff distance, the display screen(s) are automatically dimmed or turned off by an associated processor.

In particular, an aspect of the present disclosure includes a digital ocular system having the aforementioned housing, with the housing defining a housing cavity. A lens assembly that is positioned within the housing cavity includes a front lens through which a clinician, support staff, or another user of the digital ocular system views a target object, e.g., internal ocular anatomy of a patient's eye. A digital display screen is positioned within the housing cavity. The above-noted proximity sensor in this exemplary construction is connected to the housing and configured to detect the user when the user is within a predetermined standoff distance of the front lens. The same proximity sensor also outputs an electronic control signal that is indicative of the user being within the predetermined standoff distance. A processor used as part of the digital ocular system is configured to change an output state of the display screen in response to the electronic control signal, for instance by dimming the display screen or shutting it off/turning it on as needed.

Also disclosed herein is a method for controlling a digital ocular system having an eye piece, a lens assembly positioned within the eye piece, and a display screen connected to a housing. A possible implementation of the method includes detecting, using a proximity sensor connected to the housing, when a user of the digital ocular system is not within a predetermined standoff distance of a front lens of the eye piece through which the user views a target object. The method also includes transmitting an electronic sensor signal to a processor, the electronic sensor signal being indicative of the user not being within the predetermined standoff distance. An output state of the display screen is then changed via the processor in response to the electronic sensor signal.

A visualization system as set forth below includes a C mount, an ophthalmic microscope connected to the C mount, and a digital ocular system. The digital ocular system in an exemplary construction includes a housing connected to the microscope and defining therein a housing cavity, along with an eye piece having a lens assembly. The lens assembly includes a front lens through which a user of the digital ocular system views a target object. An OLED display screen is positioned within the housing cavity along an optical axis extending between the OLED display screen and the front lens.

Additionally, one or more infrared (IR) proximity sensors may be connected to the housing, e.g., an external surface thereof, and configured to detect the user when the user is within a predetermined standoff distance of the front lens, and to output an electronic sensor signal when the user is not positioned within the predetermined standoff distance. A processor is configured to change an output state of the OLED display screen in response to the electronic sensor signal, including turning off the OLED display screen after a calibrated time limit when the user is not within the predetermined standoff distance.

The above-described features and advantages and other possible features and advantages of the present disclosure will be apparent from the following detailed description when taken in connection with the accompanying drawings.

### BRIEF DESCRIPTION OF THE DRA WINGS

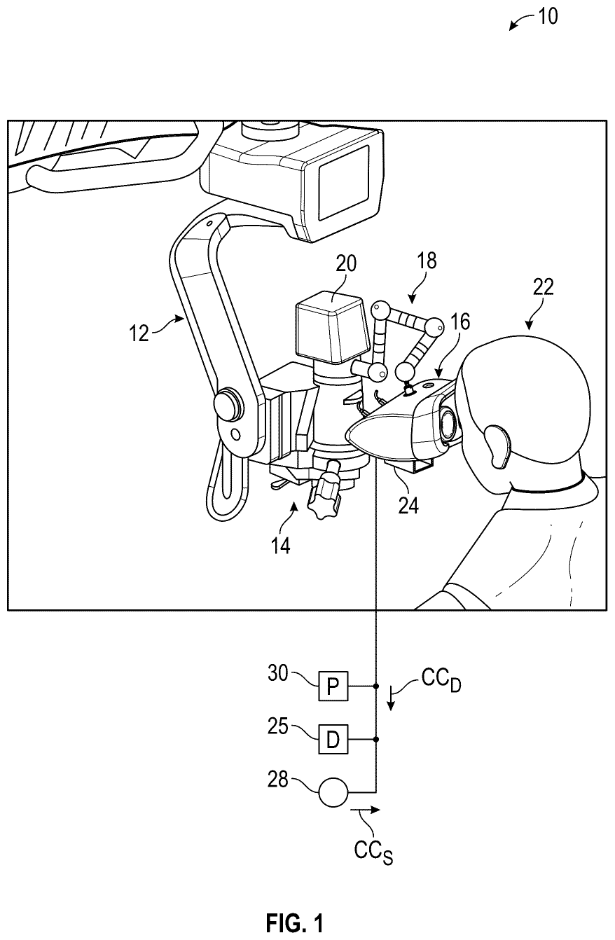

FIG. 1 illustrates an exemplary ophthalmic surgical suite having a three-dimensional (3D) visualization system equipped with digital oculars, with the digital oculars including a display screen and a proximity sensor configured in accordance with the disclosure.

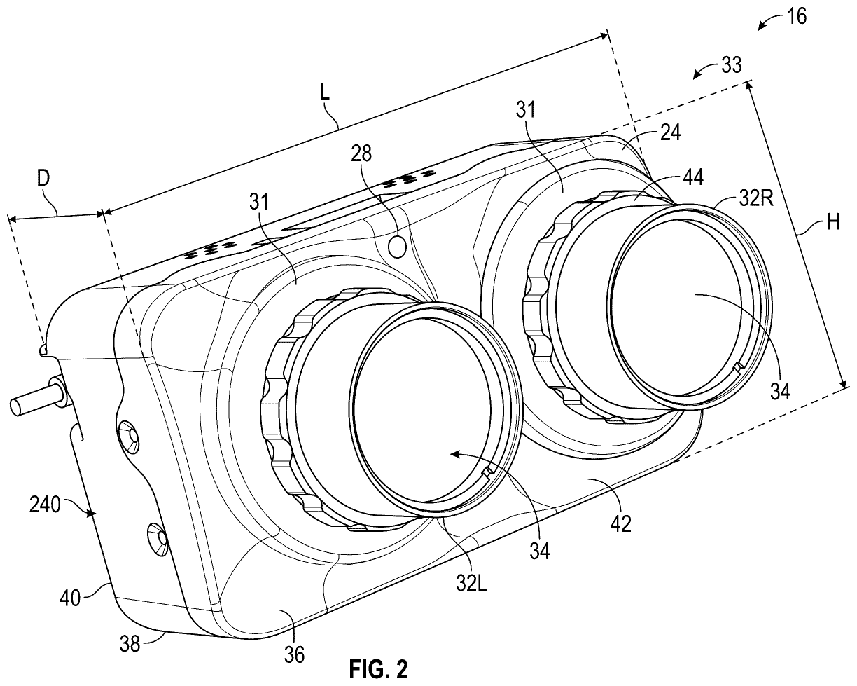

FIG. 2 is a perspective view illustration of a representative set of digital oculars having a proximity sensor in accordance with an aspect of the disclosure.

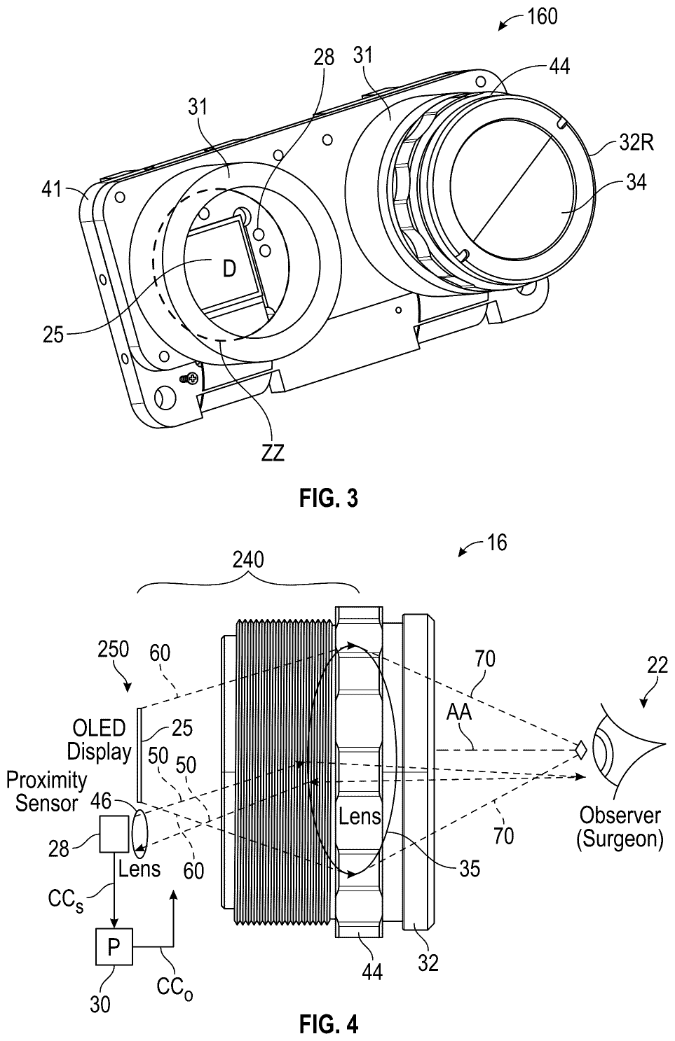

FIG. 3 illustrates an exemplary lens assembly for use with the digital oculars of FIG. 2 with one lens removed, with the proximity sensor positioned internally within the digital oculars.

FIG. 4 is a side view illustration of a digital ocular and a patient's eye depicting relative position of the proximity sensor and display screen in a possible implementation.

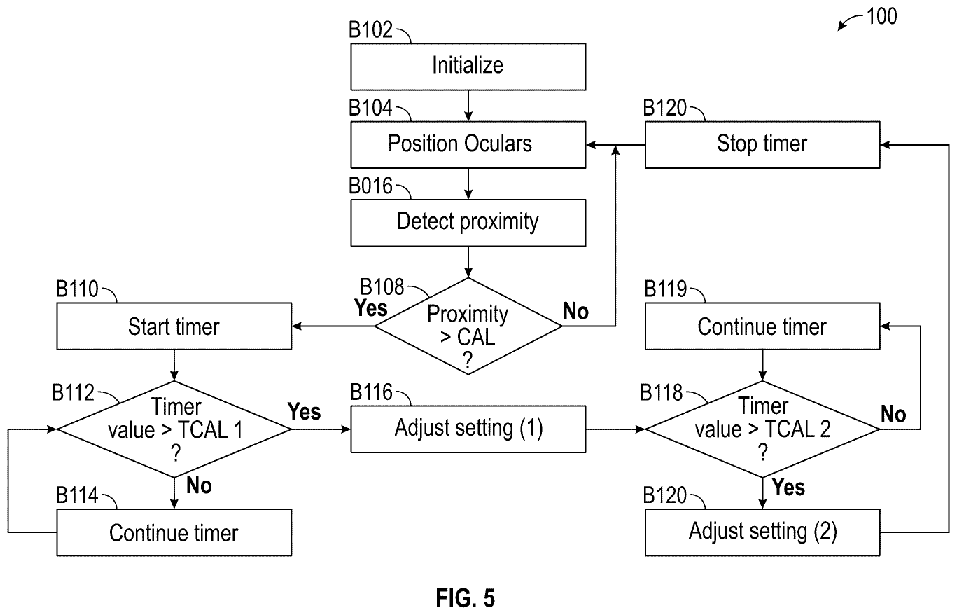

FIG. 5 is a flow chart describing a method for controlling digital oculars to prevent image burn-in in accordance with an aspect of the disclosure.

The solutions of the present disclosure may be modified or presented in alternative forms. Representative embodiments are shown by way of example in the drawings and described in detail below. However, inventive aspects of this disclosure are not limited to the disclosed embodiments. Rather, the present disclosure is intended to cover alternatives falling within the scope of the disclosure as defined by the appended claims.

### DETAILED DESCRIPTION

Referring to the drawings, wherein like reference numbers refer to like components, and beginning with FIG. 1, a visualization system 10 in accordance with the present disclosure includes a C mount 12, an ophthalmic microscope 14 connected to the C mount 12, and a digital ocular system 16 connected to the microscope 14, e.g., via an articulated bracket 18. The digital ocular system 16 is illustrated in FIG. 1 in accordance with a non-limiting exemplary configuration, with other possible embodiments being usable within the scope of the disclosure. A digital camera 20 may be connected to the microscope 14 and configured as, e.g., a charge-coupled device (CCD), a complementary metal-oxide-semiconductor (CMOS), an electron-multiplying CCD (EMCCD), or another application-suitable digital image sensor configured to output high-resolution three-dimensional (3D) image data to the digital ocular system 16 for viewing by a user 22. The user 22 may include a surgeon or other clinician/attending medical staff working within an ophthalmic surgical suite in a representative use scenario.

The digital ocular system 16 as contemplated herein includes a housing 24 that is connected to a distal end of the bracket 18. The housing 24, which may be constructed of a lightweight but sufficiently rugged material and cleanable material such as aluminum or molded plastic, defines a housing cavity 240 therewithin. As described below with reference to FIGS. 3 and 4, one or more digital display screens 25 are positioned within the housing cavity 240 as integral components of the digital ocular system 16. Such display screens 25 are susceptible to image burn-in as noted above, for instance long-term image retention, discoloration, fading, and/or ghosting of images.

To reduce or prevent effects of burn-in, therefore, the digital ocular system 16 is equipped herein with a proximity sensor 28, e.g., one or more infrared (IR) proximity sensors, e.g., a pair of IR sensors, operating in a wavelength range of about 780-1000 nm or another suitable construction. Each proximity sensor 28 outputs an electronic sensor signal (CC_{S}) to a processor (P) 30 of the visualization system 10 to inform the processor 30 as to when the user 22 is within a predetermined eye-relief distance or standoff distance of the digital ocular system 16. The processor 30 responds to the electronic sensor signal (CC_{S}) by outputting electronic control signals (CCo) to the display screen(s) (D) 25 to adjust an operating state thereof as set forth below.

As part of the present construction, the processor 30 may include, e.g., an Application Specific Integrated Circuit (ASIC), Field-Programmable Gate Array (FPGA), electronic circuits, central processing unit (CPU), microprocessor, or the like. The processor 30 could be part of a system controller (not shown) for the visualization system 10 and/or the digital ocular system 16, with wired or wireless communication occurring between the proximity sensor 28, the processor 30, and the display screen 25 over suitable transfer conductors. Thus, the state adjustments to the display screen(s) 25 may occur seamlessly without interaction with the user 22 in accordance with a method 100, an example of which is described below with reference to FIG. 5. The user 22 could also turn the digital ocular system 16 on or off as needed, e.g., using a hard switch, soft switch, voice commands, or other inputs apart from the automated state adjustment solutions provided herein.

Referring to FIG. 2, the digital ocular system 16 may include a pair of eye pieces 32L and 32R, with L and R respectively referring to the left and right eyes of the user 22 shown in FIG. 1. The digital ocular system 16 could alternatively include a single eye piece 32L or 32R in other constructions, with the digital binoculars 33 of FIG. 2 being representative. The housing 24 of FIG. 2 is generally rectangular, and thus has a length (L), height (H), and depth (D) defining the housing volume 240. Lens assemblies 34 of the eye pieces 32L and 32R are positioned within the housing cavity 240, with the lens assemblies 34 including a front lens 35 (see FIG. 4) through which the user 22 views a target object, e.g., a patient's eye (not shown) located along an optical axis of the microscope 14 of FIG. 1 when the visualization system 10 is used in an ophthalmic suite. The front lens 35 in one or more embodiments could be constructed as a complex lens with multiple elements, a single lens, or a Fresnel lens. The front lens 35 may be constructed of various application-suitable materials, including but not limited to glass or plastic, injected molded plastic, etc. As appreciated in the art, the front lens 35 may be moveable, either to enable diopter adjustments, magnification, or for another reason.

When the digital ocular system 16 is configured as the digital binoculars 33 as shown, the user 22 of FIG. 1 would view the target object using both eyes, i.e., with the surgeon's left and right eyes looking through a respective one of the front lenses 35 of FIG. 4 for three-dimensional (3D) viewing. That is, unlike 3D visualization systems which project 3D images or video of the target object onto one or more display screens or monitors and require the user 22 to don special polarized 3D glasses to properly view the displayed images, the present approach allows the user 22 to instead look directly through the eye pieces 32L and 32R in an ergonomically friendly manner without the need for such glasses, thus avoiding the potential for disorientation or vertigo in sensitive users 22. Likewise, the digital ocular system 16 allows the user 22 to freely perceive the surrounding environment, for instance to locate surgical instruments or interact with operating room staff.

Outer surfaces 36 of the housing 24 are arranged to form a generally rectangular shape, as noted above, with lateral edges 38 extending between a rear surface 40 and a front surface 42. As used herein, “front” is the particular structure or surfaces located proximate the user 22. The eye pieces 32L and 32R thus extend toward the user 22 of FIG. 1 from the front surface 42 of the housing 24. In this particular embodiment, the eye pieces 32L and 32R (together referred to as eye pieces 32) may be equipped with focusing dials 44 mounted to a circular base 31 to enable the user 22 to adjust focus or other parameters, with the front lenses 35 of FIG. 4 being located within or aft of the respective eye pieces 32L and 32R.

In the illustrated implementation, the proximity sensor 28 is connected to the front surface 42 of the housing 24 adjacent to the eye pieces 32L and 32R, in this instance midway between the eye pieces 32L and 32R. Alternative internal placement of the proximity sensor 28 is described below with reference to FIGS. 3 and 4, in which the proximity sensor 28 is instead positioned within the housing cavity 240 adjacent to the display screen 25. The placement of FIG. 2 may be sufficient for use in unobstructed operating environments. However, the presence of obstacles such as surgical drapes could potentially obstruct the proximity sensor 28, and thus the location of the proximity sensor 28 may vary with the intended application and end use.

Referring briefly to FIG. 3, an internal portion 160 of the digital ocular system 16 of FIG. 3 having a side wall 41 is illustrated with the housing 24 and the left eye piece 32L removed for clarity. As part of the digital ocular system 16, the display screen 25 is positioned within the housing cavity 240 (see FIG. 2) adjacent to the display screen 25. Transmitting an infrared beam or other sensing beam toward the user 22 could therefore include transmitting the infrared beam through the front lens 35 (see FIG. 4). The proximity sensor 28 in the illustrated configuration is connected to the housing 24 anywhere within a defined optical zone ZZ, i.e., a viewing range of the proximity sensor 28. For simplicity, the optical zone ZZ is illustrated as a circle in FIG. 3, but could be another shape or shapes depending on the proximity sensor 28 and the particular construction of the internal portion 160.

Referring now to FIG. 4, the proximity sensor 28 of FIGS. 1-3 is configured to detect the user 22 of FIG. 1 when the user 22 is within the predetermined standoff distance (AA) of the front lens 35. In a possible implementation, the standoff distance (AA) is about 25-30 millimeters (mm). However, the standoff distance (AA) is application-specific, and may be larger or smaller than 25-30 mm in other embodiments. The proximity sensor 28 outputs the electronic sensor signal (CC_{S}) indicative of proximity of an object, in this case the user 22, within the field-of-view of the proximity sensor 28. When the proximity sensor 28 detects that the user 22 is not within the standoff distance, i.e., the user 22 is not actively looking through the eye pieces 32L and 32R of FIG. 2, the value of the electronic sensor signal (CC_{S}) will change. The processor 30 responds to this change by outputting the electronic control signal (CC_{E}) to the display screen(s) 25. The electronic control signal (CC_{E}) is indicative of the user 22 being within (or not within) the predetermined standoff distance (AA). As noted above, the processor 30 is configured to change an output state of the display screen 25 in response to the value of the electronic control signal (CC_{E}), such as by temporarily dimming or shutting off the display screen 25 to prevent burn-in.

In the illustrated configuration of FIG. 4, the display screen 25 is an organic light-emitting diode (OLED) display screen 250 positioned within the housing cavity 240 along an optical axis extending between the display screen 25 and the front lens 35. Other embodiments of the display screen 25 may be used within the scope of the disclosure, with OLED being just one possible construction susceptible to the problem of burn-in as set forth above. An optional focusing lens 46 may be positioned near the proximity sensor 28 in one or more embodiments to help focus a sensing beam 50 from the proximity sensor 28. However, other embodiments can forego use of the focusing lens 46 when the proximity sensor 28 is appropriately positioned, e.g., when placed within the optical zone ZZ of FIG. 3. Images on each display screen 25 are projected toward the user 22, as indicated by projection lines 60, which are directed to the user 22 via the front lens 35 as a projected image 70. Thus, the user 22 is able to forego use of 3D glasses in lieu of direct viewing via the digital ocular system 16.

Referring to FIG. 5, a method 100 for controlling the digital ocular system 16 having the lens assembly and display screen(s) 25 connected to the housing 24 of FIG. 1 is described in terms of discrete process segments or blocks for illustrative clarity. The constituent blocks of the method 100 may be performed by the processor 30 when the user 22 performs a 3D visualization technique using the digital ocular system 16.

Beginning with block B102, the method 100 commences with initialization of the visualization system 10 of FIG. 1. A patient is positioned below the microscope 14 and the user 22 powers on the various components of the visualization system 10, including the microscope 14, digital camera 20, and digital ocular system 16. The method 100 then proceeds to block B104.

Block B104 of the method 100 may include manually or automatically positioning the digital ocular system 16 to facilitate viewing by the user 22 using the now-initialized visualization system 10 of FIG. 1. During eye surgery, for instance, the user 22 could peer through the eye pieces 32R and 32L of FIG. 2 to view magnified images through the front lenses 35. The user 22 does so in conjunction with performing other surgical tasks, and therefore the user 22 could periodically use the digital ocular system 16 when performing such tasks. The method 100 proceeds to block B106 as the procedure progresses.

Block B106 of FIG. 5 entails detecting, via the proximity sensor 28 of FIGS. 1-4, a proximity of the user 22 to the digital ocular system 16. Block B106 therefore determines when the user 22 is within the predetermined standoff distance (AA) of the front lens 35 (FIG. 4) of the lens assembly through which the user 22 views a target object, in this case ocular anatomy of a patient's eye (not shown). Depending on the construction of the proximity sensor 28, block B106 could include directing the sensor beam 50 of FIG. 4 toward the user 22, either directly or through the intervening focusing lens 46 in different embodiments. The output of the proximity sensor 28, i.e., the electronic sensor signal (CC_{S}), could be a voltage signal having a value indicative of the distance between the user 22 and the proximity sensor 28. Detecting when the user 22 of the digital ocular system 16 is within the predetermined standoff distance (AA) of the front lens 35 could include transmitting an infrared beam toward the user 22 via the proximity sensor 28 when the proximity sensor 28 is configured as an infrared sensor.

The proximity sensor 28 may include a pair of proximity sensors 28, such that the digital ocular system 16 is configured as a set of digital binoculars as noted above. In such a case, the lens assembly would include a pair of lens assemblies and the display screen 25 includes a pair of display screens 25. Detecting when the user 22 is within the predetermined standoff distance (AA) of the front lens 35 is thus accomplished using the pair of proximity sensors 28 in such an embodiment. The method 100 proceeds to block B108 as the proximity sensor 28 performs its proximity sensing functions.

At block B108, the processor 30 of FIGS. 1 and 4 processes the electronic sensor signal (CC_{S}) and compares the same to a predetermined or calibrated standoff distance (AA) of FIG. 4, e.g., 25-30 mm or another suitable value or range. The method 100 proceeds to block B110 when the proximity sensor 28 detects that the user 22 is within the calibrated standoff distance. The method 100 instead returns to block B104 when the user 22 remains within the calibrated standoff distance.

At block B110, the processor 30 may start a timer in response to the determination at block B108 that the user 22 has moved beyond the calibrated standoff distance (AA) of the proximity sensor 28. Block B110 could be implemented to provide a suitable delay to allow for transient movement of the user 22 just beyond the range of the proximity sensor 28, for instance when the user 22 reaches for a surgical tool or briefly pulls away from the eye pieces 32L and 32R of FIG. 2 to speak to attending staff. The method 100 thereafter proceeds to block B112.

Block B112 includes determining whether an elapsed time value of the timer from block B110 has reached a calibrated time limit (T_{CAL1}), e.g., 5-10 seconds or another application-suitable amount of time. The method 100 continues to block B114 when the timer has not yet reached the calibrated time limit, and to block B116 in the alternative once the calibrated time limit has elapsed.

Block B114 includes continuing the timer initiated at block B110. The method 100 returns to block B112 as the timer continues to count upward toward the first calibrated time limit (T_{CAL1}).

Block B116 of FIG. 5 includes transmitting the electronic sensor signal (CC_{S}) to the processor 30, with the electronic sensor signal (CC_{S}) indicative of the user 22 being outside of the predetermined standoff distance (AA). In response to the electronic sensor signal (CC_{S}), the method 100 may include changing an output state of the display screen(s) 25 via the processor 30. For example, the processor 30 may adjust a brightness level of the display screen(s) 25 of FIGS. 1, 3, and 4, e.g., an OLED screen as noted above. For example, the processor 30 could dim the display screen(s) 25 after the first calibrated time limit of block B112 has elapsed. The method 100 then proceeds to block B118.

At block B118, the processor may next determine whether an elapsed time value of the timer from block B110 has reached a second calibrated time limit (T_{CAL2}), e.g., 10-15 seconds or another application-suitable amount of time. The method 100 continues to block B119 when the timer has not yet reached the second calibrated time limit, and to block B120 in the alternative once the second calibrated time limit has elapsed.

At block B119, the timer continues to count. The method 100 continues to block B118 as this occurs.

Block B120 is reached when the timer initiated at block B110 reaches the second calibrated time limit (T_{CAL2}), whereupon the processor 30 adjusts one or more settings of the display screen(s) 25 of FIGS. 1, 3, and 4. For example, the processor 30, having already dimmed the display screen(s) 25 at block B116 after the first calibrated time limit (T_{CAL1}), may turn off the display screen(s) 25 in block B120. While the display screen(s) 25 could also be turned off immediately when the user 22 moves out of proximity of the front lens 35 of the digital ocular system 16, the use of two or possibly more time limits would allow for a more gradual control response, one that could be less distracting to the user 22.

Embodiments shown in the drawings or the characteristics of various embodiments mentioned in the present description are not necessarily to be understood as being independent of each other. It is possible that each of the characteristics described in a given embodiment could be combined with one or more other desired characteristics from other embodiments, resulting in other embodiments not described in words or by reference to the drawings.

As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Certain terminology may be used in the following description for the purpose of reference only, and thus are not intended to be limiting. For example, terms such as “above” and “below” refer to directions in the drawings to which reference is made. Terms such as “front,” “back,” “fore,” “aft,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

Accordingly, such other embodiments fall within the framework of the scope of the appended claims. The detailed description and the drawings are supportive and descriptive of the disclosure, but the scope of the disclosure is defined solely by the claims. While various modes for carrying out the claimed disclosure have been described in detail, various alternative designs and embodiments exist for practicing the disclosure defined in the appended claims.

What we claim is:

1. A digital ocular system, comprising:
a housing defining a housing cavity;
a lens assembly positioned within the housing cavity, the lens assembly including an eye piece having a front lens through which a user of the digital ocular system views a target object;
a display screen positioned within the housing cavity;
a proximity sensor connected to the housing, wherein the proximity sensor is configured to detect when the user is within a predetermined standoff distance of the front lens, and to output an electronic sensor signal that is indicative of the user being outside of the predetermined standoff distance; and
a processor configured to adjust an output state of the display screen via a display control signal in response to the electronic sensor signal.

2. The digital ocular system of claim 1, wherein the digital ocular system is configured as digital binoculars in which the eye piece includes a pair of eye pieces, the lens assembly includes a pair of lens assemblies within the pair of eye pieces, and the display screen includes a pair of display screens.

3. The digital ocular system of claim 1, wherein the display screen includes an organic light-emitting diode screen, and wherein the digital ocular system is configured to connect to an ophthalmic microscope.

4. The digital ocular system of claim 1, wherein the proximity sensor includes an infrared sensor.

5. The digital ocular system of claim 1, wherein the proximity sensor is connected to a surface of the housing and adjacent to the eye piece.

6. The digital ocular system of claim 1, wherein the proximity sensor is positioned within the housing cavity and adjacent to the display screen.

7. The digital ocular system of claim 1, wherein the processor is configured to change the output state of the display screen in response to the electronic sensor signal by turning off the display screen when the user is not within the predetermined standoff distance of the front lens.

8. The digital ocular system of claim 7, wherein the processor is configured to change the output state of the display screen in response to the electronic sensor signal by dimming the display screen when the user is not within the predetermined standoff distance of the front lens, and then turning off the display screen after a calibrated time limit.

9. A method for controlling a digital ocular system having an eye piece, a lens assembly positioned within the eye piece, and a display screen connected to a housing, comprising:
detecting, using a proximity sensor connected to the housing, when a user of the digital ocular system is not within a predetermined standoff distance of a front lens of the eye piece through which the user views a target object;
transmitting an electronic sensor signal to a processor, the electronic sensor signal being indicative of the user not being within the predetermined standoff distance; and
changing an output state of the display screen via the processor in response to the electronic sensor signal.

10. The method of claim 9, wherein the proximity sensor includes a pair of proximity sensors, the digital ocular system is configured as digital binoculars in which the eye piece includes a pair of eye pieces, the lens assembly includes a pair of lens assemblies within the pair of eye pieces, and the display screen includes a pair of display screens, and wherein detecting when the user is not within the predetermined standoff distance of the front lens is accomplished using the pair of proximity sensors.

11. The method of claim 9, wherein the display screen includes an organic light-emitting diode (OLED) screen, and wherein changing the output state of the display screen includes changing a brightness level of the OLED screen.

12. The method of claim 9, wherein the proximity sensor includes an infrared sensor, and wherein detecting when the user of the digital ocular system is not within the predetermined standoff distance of the front lens includes transmitting an infrared beam toward the user via the infrared sensor.

13. The method of claim 12, wherein the proximity sensor is positioned within the housing adjacent to the display screen, and wherein transmitting an infrared beam toward the user includes transmitting the infrared beam through the front lens.

14. The method of claim 9, wherein changing the output state of the display screen in response to the electronic sensor signal includes turning off the display screen when the user is not within the predetermined standoff distance of the front lens.

15. The method of claim 14, wherein changing the output state of the display screen in response to the electronic sensor signal includes dimming the display screen when the user is not within the predetermined standoff distance of the front lens for a calibrated time limit, and thereafter turning off the display screen after reaching the calibrated time limit.

16. A visualization system, comprising:
an ophthalmic microscope; and
a digital ocular system comprising:
a housing connected to the microscope and defining therein a housing cavity;
an eye piece having a lens assembly, the lens assembly including a front lens through which a user of the digital ocular system views a target object;
an organic light-emitting diode (OLED) display screen positioned within the housing cavity along an optical axis extending between the OLED display screen and the front lens;
an infrared proximity sensor connected to the housing, wherein the infrared proximity sensor is configured to detect the user when the user is within a predetermined standoff distance of the front lens, and to output an electronic sensor signal when the user is not positioned within the predetermined standoff distance; and
a processor configured to change an output state of the OLED display screen in response to the electronic sensor signal, including turning off the OLED display screen after a calibrated time limit when the user is not within the predetermined standoff distance.

17. The visualization system of claim 16, wherein the digital ocular system is configured as digital binoculars in which the lens assembly includes a pair of lens assemblies and the OLED display screen includes a pair of OLED display screens.

18. The visualization system of claim 17, wherein the infrared proximity sensor includes is connected to an external surface of the housing and adjacent to the front lens.

19. The visualization system of claim 17, wherein the proximity sensor includes a pair of infrared proximity sensors each positioned within the housing cavity and adjacent to a respective one of the OLED display screens.

20. The visualization system of claim 16, wherein the processor is configured to change the output state of the OLED display screen in response to the electronic sensor signal by dimming the OLED display screen, and thereafter turning off the display screen after the OLED display screen has been dimmed for a calibrated time limit.
